(19) United States
(12) Patent Application Publication
(10) Pub. No.: US 2023/0139668 A1
(43) Pub. Date: May 4, 2023

(54) CONNECTOR SYSTEM AND METHOD FOR CONNECTING AN ELECTROSURGICAL INSTRUMENT TO AN ELECTROSURGICAL GENERATOR

(71) Applicant: Covidien LP, Mansfield, MA (US)

(72) Inventor: James E. Thompson, Frederick, CO (US)

(21) Appl. No.: 17/960,651

(22) Filed: Oct. 5, 2022

Related U.S. Application Data
(60) Provisional application No. 63/274,599, filed on Nov. 2, 2021.

(51) Int. Cl. A61B 17/32; A61B 18/14

(52) U.S. Cl. CPC A61B 17/32; A61B 18/1442; A61B 2017/320095

(57) ABSTRACT

A connector system for connecting an electrosurgical instrument to an electrosurgical generator includes a connector plug coupled to the electrosurgical instrument and a plug receptacle defined in the electrosurgical generator. The connector plug includes a connector housing and a connector pin extending from the connector housing, and the connector pin includes an elongated body and a protrusion extending laterally outwardly from the elongated body. The plug receptacle includes an electrical connector disposed therein, and the electrical connector includes a substrate having a notch defined therein and an electrical contact region disposed thereon. The connector plug is removably insertable into the plug receptacle such that the protrusion of the connector pin is positioned within the notch of the electrical connector and the elongated body of the connector pin contacts the electrical contact region of the electrical connector.

### CROSS-REFERENCE TO RELATED APPLICATIONS

This application claims the benefit of, and priority to, U.S. Provisional Patent Application No. 63/274,599, filed on Nov. 2, 2021, the entire contents of which are hereby incorporated herein by reference.

### FIELD

This disclosure generally relates to electrosurgical instruments, and more particularly, to connector systems for selectively connecting electrosurgical instruments to an electrosurgical generator.

### BACKGROUND

Electrosurgical instruments are employed by surgeons to perform a variety of endoscopic, robotic, and/or open surgical procedures for treating tissue including cutting, coagulating, cauterizing, desiccating, and/or sealing tissue. High frequency electrical power, typically radio frequency (RF) power or energy, is produced by an electrosurgical generator and applied to the tissue by the electrosurgical instrument. The electrosurgical instrument is operated and manipulated by a surgeon or a surgical robot to perform the desired electrosurgical procedure.

The connection between the electrosurgical instrument and the electrosurgical generator is typically removable, such that the electrosurgical instrument may be connected and disconnected from the electrosurgical generator over the course of a single procedure, over a number of electrosurgical procedures, and/or a first electrosurgical instrument configured for performing a first type of surgical procedure may be connected and disconnected from the electrosurgical generator and a second electrosurgical instrument configured for performing a second type of surgical procedure may be connected to the electrosurgical generator.

### SUMMARY

This disclosure relates to connector systems for connecting electrosurgical instruments to an electrosurgical generator. The electrosurgical instruments include a connector which provides a reliable and easily insertable and removable, physical and electrical connection to a connector of the electrosurgical generator. In aspects, the connector systems are designed to reduce wear on the conductive surfaces of the connectors of the electrosurgical instrument and the electrosurgical generator thereby increasing reliability of the connection between the electrosurgical instrument and the electrosurgical generator. In some aspects, the connector systems are designed to encode information on the connector of the electrosurgical instrument and communicate that information to the electrosurgical generator for identifying the electrosurgical instrument connected to the electrosurgical generator, detecting if incorrect or incomplete insertion of the connector of the electrosurgical instrument into the connector of the electrosurgical generator is made, and/or allowing for make-before-break or make-before-make functions to be employed, thereby increasing electrosurgical instrument and electrosurgical generator reliability.

In aspects, this disclosure provides a connector system for connecting an electrosurgical instrument to an electrosurgical generator. The connector system includes a connector plug coupled to the electrosurgical instrument and a plug receptacle defined in the electrosurgical generator. The connector plug includes a connector housing and a connector pin extending from the connector housing, and the connector pin includes an elongated body and a protrusion extending laterally outwardly from the elongated body. The plug receptacle includes an electrical connector disposed therein, and the electrical connector includes a substrate having a notch defined therein and an electrical contact region disposed thereon. The connector plug is removably insertable into the plug receptacle such that the protrusion of the connector pin is positioned within the notch of the electrical connector and the elongated body of the connector pin contacts the electrical contact region of the electrical connector.

In some aspects, the protrusion of the connector pin is positioned between proximal and distal segments of the elongated body. In certain aspects, the notch defined in the electrical connector is proximal to the electrical contact region and the distal segment of the connector pin contacts the electrical contact region when the connector plug is inserted in the plug receptacle.

In some aspects, the notch and the electrical contact region of the electrical connector is defined on a first surface of the substrate. In certain aspects, the notch and the electrical contact region are disposed on a planar segment of the first surface, and the first surface includes a tapered segment disposed proximal to the planar segment.

The protrusion of the connector pin and the notch of the electrical connector may have complementary geometries. The elongated body of the connector pin may be formed from a conductive material and the protrusion may be formed from a non-conductive material. The electrical connector may be a printed circuit board. The connector pin of the connector plug may be one of a plurality of connector pins and the electrical connector of the plug receptacle may be one of a plurality of electrical connectors, and the plurality of connector pins may be aligned with the plurality of electrical connectors.

In some aspects, during insertion of the connector plug into the plug receptacle, the protrusion of the connector pin is configured to slide along the substrate of the electrical connector such that the elongated body of the connector pin does not contact the electrical connector until the protrusion aligns with the notch of the electrical connector.

In aspects, this disclosure provides an electrosurgical system including an electrosurgical instrument including a connector plug and an electrosurgical generator including a plug receptacle. The connector plug includes a connector housing and a connector pin extending from the connector housing, and the connector pin includes an elongated body and a protrusion extending laterally outwardly from the elongated body. The plug receptacle includes an electrical connector disposed therein, and the electrical connector includes a substrate having a notch defined therein and an electrical contact region disposed thereon. The connector plug is removably insertable into the plug receptacle such that the protrusion of the connector pin is positioned within the notch of the electrical connector and the elongated body of the connector pin contacts the electrical contact region of the electrical connector.

In some aspects, the protrusion of the connector pin is positioned between proximal and distal segments of the elongated body. In certain aspects, the notch defined in the electrical connector is proximal to the electrical contact region and the distal segment of the connector pin contacts the electrical contact region when the connector plug is inserted in the plug receptacle.

In some aspects, the notch and the electrical contact region of the electrical connector is defined on a first surface of the substrate. In certain aspects, the notch and the electrical contact region are disposed on a planar segment of the first surface, and the first surface includes a tapered segment disposed proximal to the planar segment.

The protrusion of the connector pin and the notch of the electrical connector may have complementary geometries. The elongated body of the connector pin may be formed from a conductive material and the protrusion may be formed from a non-conductive material. The electrical connector may be a printed circuit board. The connector pin of the connector plug may be one of a plurality of connector pins and the electrical connector of the plug receptacle may be one of a plurality of electrical connectors, and the plurality of connector pins may be aligned with the plurality of electrical connectors.

In some aspects, during insertion of the connector plug into the plug receptacle, the protrusion of the connector pin is configured to slide along the substrate of the electrical connector such that the elongated body of the connector pin does not contact the electrical connector until the protrusion aligns with the notch of the electrical connector.

In aspects, this disclosure provides a method of using an electrosurgical system including: connecting an electrosurgical instrument to an electrosurgical generator by inserting a connector plug of the electrosurgical instrument into a plug receptacle of the electrosurgical generator such that a protrusion of a connector pin of the connector plug is positioned within a notch defined in an electrical connector disposed within the plug receptacle, and an elongated body of the connector pin contacts an electrical contact region of the electrical connector.

The method may further include: inserting an end effector of the electrosurgical instrument into tissue; and actuating an energy activation actuator to control a supply of energy from the electrosurgical generator to the end effector of the electrosurgical instrument.

The details of one or more aspects of this disclosure are set forth in the accompanying drawings and the description below. Other aspects, as well as features, objects, and advantages of the aspects described in this disclosure will be apparent from the description and drawings, and from the claims.

### BRIEF DESCRIPTION OF DRAWINGS

Various aspects of this disclosure are described hereinbelow with reference to the drawings, which are incorporated in and constitute a part of this specification, wherein:

FIG. 1 is a perspective view of an electrosurgical system including an electrosurgical instrument and an electrosurgical generator in accordance with aspects of the disclosure;

FIG. 2 is a perspective view of a connector system of the electrosurgical assembly of FIG. 1, showing a connector plug of the electrosurgical instrument connected to a plug receptacle of the electrosurgical generator;

FIG. 3A is a partial, side view of a connector pin of the connector plug of FIG. 2 and an electrical connector disposed within the plug receptacle of FIG. 2, shown prior to insertion of the connector plug into the plug receptacle;

FIG. 3B is a partial, side view of the connector pin of FIG. 3A contacting the electrical connector of FIG. 3A during insertion of the connector plug into the plug receptacle;

FIG. 3C is a partial, side view of the connector pin of FIG. 3B physically and electrically connected to the electrical connector of FIG. 3B;

FIG. 4 is a perspective view of another electrosurgical instrument suitable for use with the electrosurgical generator of FIG. 1; and

FIG. 5 is a perspective view of yet another electrosurgical instrument suitable for use with the electrosurgical generator of FIG. 1.

### DETAILED DESCRIPTION

Aspects of this disclosure will now be described in detail with reference to the drawing figures wherein like reference numerals identify similar or identical elements. It should be understood that various components of the disclosure, such as those plainly numbered, correspond to components of the disclosure prime or double prime numbered, such that redundant explanation of similar components need not be repeated herein. Throughout this description, the term “proximal” refers to a portion of a structure, or component thereof, that is closer to an operator (whether a human surgeon, other medical professional, or a surgical robot), and the term “distal” refers to a portion of the structure, or component thereof, that is farther from the operator. It should be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

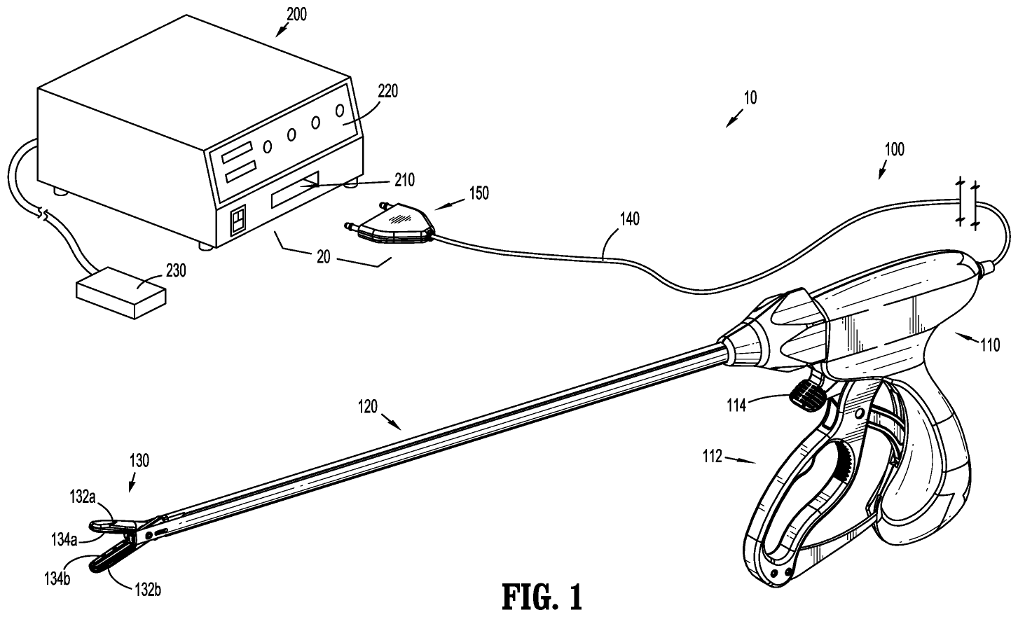

FIG. 1 illustrates an electrosurgical system 10 including an electrosurgical instrument 100 and an electrosurgical generator 200. The electrosurgical instrument 100 is electrically connectable to the electrosurgical generator 200. The electrosurgical instrument 100 is shown in the form of an endoscopic forceps and includes a housing 110 including a handle assembly 112, an elongated shaft 120 extending distally from the housing 110, and an end effector 130 disposed at a distal end of the elongated shaft 120. Various internal operating components of the electrosurgical instrument 100 are stored within the housing 110, the elongated shaft 120, and the end effector 130, and the end effector 130 is configured to treat tissue of a patient. The end effector 130 includes first and second jaws 132a, 132b that cooperate to rotate, articulate, grasp, seal, and/or divide tissue. The first and second jaws 132a, 132b include respective sealing plates 134a, 134b which communicate electrosurgical energy through tissue held therebetween. The electrosurgical instrument 100 also includes a cable 140 having a connector plug 150 at a distal end thereof which connects the electrosurgical instrument 100 to the electrosurgical generator 200. The cable 140 is internally divided into cable leads which each transmit electrosurgical energy through their respective feed paths through the electrosurgical instrument 100 and to the end effector 130 when the connector plug 150 is coupled to the electrosurgical generator 200. Control leads may also be provide to communicatively couple electrosurgical instrument 100 and electrosurgical generator 200.

The electrosurgical generator 200 is source of electrosurgical energy, such as radiofrequency energy, which is connectable to the electrosurgical instrument 100 for transmitting the electrosurgical energy therethrough. The electrosurgical generator 200 includes a plug receptacle 210 configured to receive the connector plug 150 of the electrosurgical instrument 100, a control panel 220 that provides an interface for the user, and may include an energy activation actuator 230, such as a foot pedal, for enabling selective activation of electrosurgical energy from the electrosurgical generator 200 to the electrosurgical instrument 100. The electrosurgical generator 200 may be configured to be selectively set (e.g., via the control panel 220) to an energy frequency or signal appropriate for the attached electrosurgical instrument 100 and/or the electrosurgical procedure being performed, or the electrosurgical generator 200 may be configured to automatically configure itself to transmit a particular energy frequency or signal depending, for example, on the electrosurgical instrument 100 connected thereto. It should be understood that the electrosurgical generator 200 may use different sources of energy such as, for example, high frequency, ultrasound, thermal, microwave, light, or molecular resonance, utilizing different techniques (e.g., monopolar or bipolar) and, in some aspects, other medical or surgical delivery sources may be used, such as, for example, lasers, hydro dissectors, or aspirators.

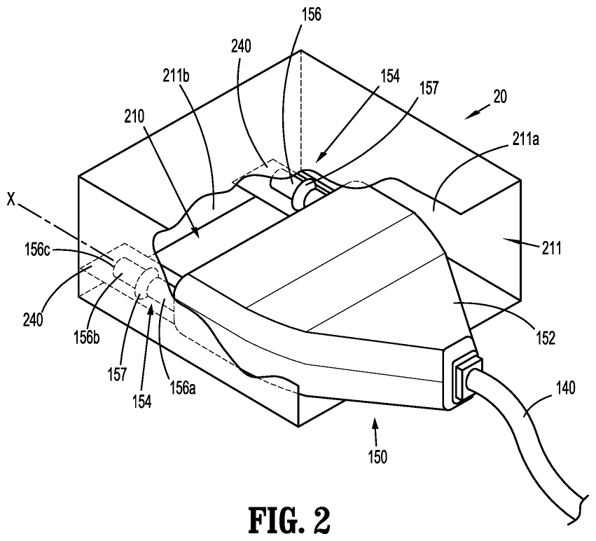

FIGS. 1 and 2 illustrate a connector system 20 of the electrosurgical system 10. The connector system 20 includes the connector plug 150 of the electrosurgical instrument and the plug receptacle 210 of the electrosurgical generator 200. The connector plug 150 is positionable within the plug receptacle 210 and configured to mechanically engage the plug receptacle 210 to create an electrical connection between the electrosurgical instrument 150 and the electrosurgical generator 200 for delivery of the electrosurgical energy.

As seen in FIG. 2, the connector plug 150 includes a connector housing 152 and connector pins 154 extending distally from the connector housing 152. Each of the connector pins 154 includes an elongated body 156 extending along a longitudinal axis “X.” The elongated body 156 has a generally cylindrical shape, and includes a proximal segment 156a and a distal segment 156b terminating at a distal tip 156c. A protrusion 157 extends laterally (in aspects, radially) outwardly from the elongated body 156 and is positioned between the proximal and distal segments 156a, 156b of the elongated body 156 such that the portion of the elongated body 156 including the protrusion 157 has a larger diameter or dimension than the rest of the elongated body 156. The protrusion 157 may be a bump, ridge, or other projection having a generally rounded or curve profile that extends partially or entirely around the elongated body 156 such that the protrusion 157 is configured to contact and engage an electrical connector 240 of the plug receptacle 210, as described in further detail below.

In some aspects, the protrusion 157 is integrally formed with the elongated body 156 (e.g., stamping or injection molding) and, in some aspects, the protrusion 157 is secured to the elongated body 156 (e.g., electroplating or over-molding). The elongate body 156 of the connector pin 156 is formed from a conductive material, such as gold, copper, or stainless steel, and is coupled to a cable lead of the cable 140 such that when electrical connection is made with the electrosurgical generator 200, energy is transmitted through the electrosurgical instrument 100. In some aspects, the protrusion 157 is formed from the same or different conductive material as the elongated body 156, however, the protrusion 157 may be formed from any material (e.g., a non-conductive material, such as a polymer or plastic) of sufficient strength and rigidity to withstand frictional wear and tear during insertion and removal of the connector plug 150 into and out of the plug receptacle 210.

While the elongated body 156 of the connector pins 154 are shown having a substantially cylindrical shape, it should be understood that the elongated body 156 may have other shapes, such as substantially rectangular blades. Further, while the connector plug 150 is shown including two connector pins 154, it should be understood that the number of connector pins 154, as well as the size, shape, and/or placement of the connector pins 154 may vary.

The plug receptacle 210 of the electrosurgical generator 200 defines a cavity 211 therein that is sized and shaped to receive the connector plug 150. The cavity 211 includes a proximal portion 211a sized and shaped to receive the connector housing 152 of the connector plug 150 in a friction fit manner, and a distal portion 211b having electrical connectors 240 extending therein that are each aligned and configured to interface with a respective connector pin 154 of the connector plug 150. The plug receptacle 210 of the electrosurgical generator 200 may be configured to receive or connect to connector plugs of various electrosurgical instruments, and/or may be configured to receive or connect to electrosurgical instruments which includes more or less connector pins.

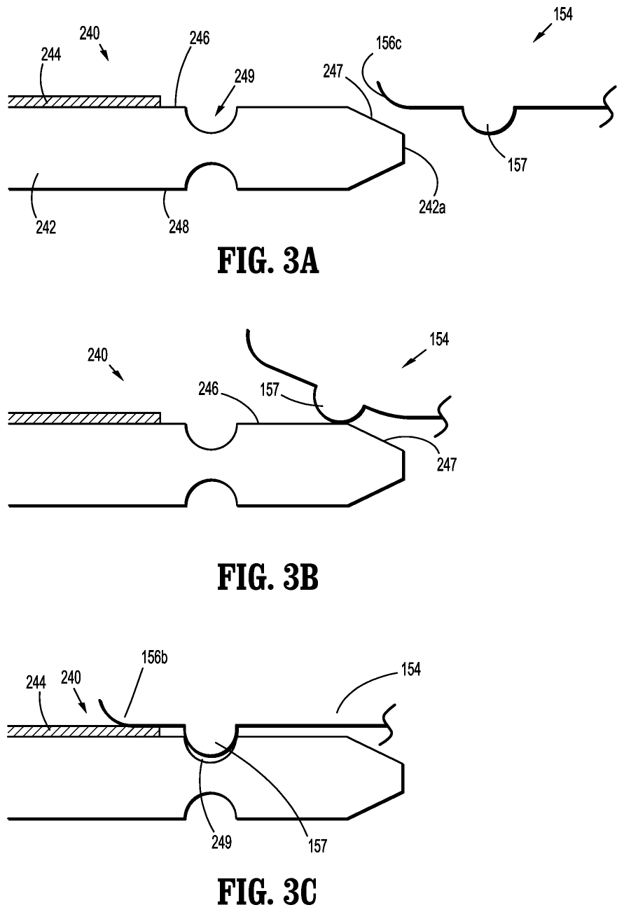

As shown in FIG. 3A, the electrical connector 240 is shown in the form of a printed circuit board including a body or substrate 242 supporting an electrical contact region 244 thereon (e.g., a terminal end of a conductive trace defined through the substrate 242). The electrical contact region 244 is formed from a conductive material, such as copper, and the substrate 242 is formed from a non-conductive material, such as a polymer or a ceramic. The electrical connector 240 enables communication between the electrosurgical generator 200 and the electrosurgical instrument 100 and transmittal of electrosurgical energy from the electrosurgical generator 200 to the electrosurgical instrument 100. In some aspects, the electrical connector 240 includes electronic components supporting an encoding scheme that identifies and/or recognizes the presence and/or type of a connector pin 154 and communicates information about the electrosurgical instrument 100 and/or the connection therewith to the electrosurgical generator 200. In certain aspects, the connector system 20 enables communication between sensor(s) of the electrosurgical instrument 100 and/or electrosurgical generator 200.

The substrate 242 of the electrical connector 240 has a generally planar profile and includes opposed first and second surfaces 246, 248. The first surface 246 includes a sloped or tapered segment 247 extending to a proximal end 242a of the substrate 242, a notch 249 defined in the first surface 246 distal to and in axially spaced relation relative to the tapered segment 247, and the electrical contact region 244 disposed distal to and in spaced relation relative to the notch 249. The notch 249 has a complementary geometry with the protrusion 157 of the connector pin 154. The second surface 248 of the substrate 242 may be a mirror image of the first surface 246.

During insertion of the connector plug 150 into the plug receptacle 210, as seen in FIG. 3A, the distal tip 156c of the connector pin 154 slides adjacent to the proximal end 242a of the substrate 242 of the electrical connector 240 until the protrusion 157 of the connector pin 154 contacts the electrical connector 240. The protrusion 157 initially slides against the tapered segment 247 of the first surface 246 and deflects the connector pin 154 laterally outwardly away from its biased position, as shown in FIG. 3B, as the protrusion 157 moves distally along the first surface 246 of the electrical connector 240. The connector pin 154 continues its distal movement along the first surface 246 of the electrical connector 240 until the protrusion 157 of the connector pin 154 aligns with and drops down into the notch 249 of the electrical connector 240 whereby the connector pin 154 returns to its biased position, as shown in FIG. 3C, such that the distal segment 156b of the connector pin 154 contacts the electrical contact region 244 of the electrical connector 240 thereby making a physical connection between the connector pin 154 and the electrical connector 240 and enabling communication between the electrosurgical instrument 100 and the electrosurgical generator 200.

The tapered segment 247 and the notch 249 of the electrical connector 240 and the protrusion 157 of the connector pin 154 work together to ensure contact of the distal segment 156b of the connector pin 154 with the electrical contact region 244 of the electrical connector 240 once the connector plug 150 is fully inserted into the plug receptacle 210, while minimizing or preventing wear on these critical conductive areas (e.g., the distal segment 156b of the connector pin 154 and the electrical contact region 244 of the electrical connector 240) during the insertion and removal processes. In some aspects, the distal segment 156b of the connector pin 154 has limited contact and, in certain aspects, no contact, with any portion of the electrical connector 240 until the protrusion 157 enters the notch 249 and the distal segment 156b is deflected into contact with the electrical contact region 244 of the electrical connector 240 (e.g., only the protrusion 157 contacts the electrical connector 240 during sliding of the connector plug 150 into the plug receptacle 210).

Referring also to FIG. 1, during operation of the electrosurgical instrument 10, with the connector plug 150 engaged within the plug receptacle 210 of the electrosurgical generator 200 and the end effector 130 positioned within tissue of a patient, a user controls the supply of energy to the end effector 130 of the electrosurgical instrument 100 by depressing an energy activation actuator 114 supported on the electrosurgical instrument 100 or an energy activation actuator 230 coupled to the electrosurgical generator 200. Specifically, the connector pins 154 are electrically coupled to the electrical contact regions 244 in the plug receptacle 240 thereby providing a path for the electrosurgical energy through the electrosurgical instrument 100 and to tissue of a patient via the end effector 130.

After the user is finished using the electrosurgical instrument 100, the connector plug 150 is removed from the plug receptacle 210. When the connector plug 150 is removed, the protrusion 157 of the connector pin 154 slides out of the notch 249 defined in the electrical connector 240, across the first surface 246 of the substrate 242, and along the tapered surface 247 thereby separating the connector plug 150 from the plug receptacle 210 without causing wear on the distal segment 156b of the connector pin 154 or the electrical contact region 244 of the electrical connector 240.

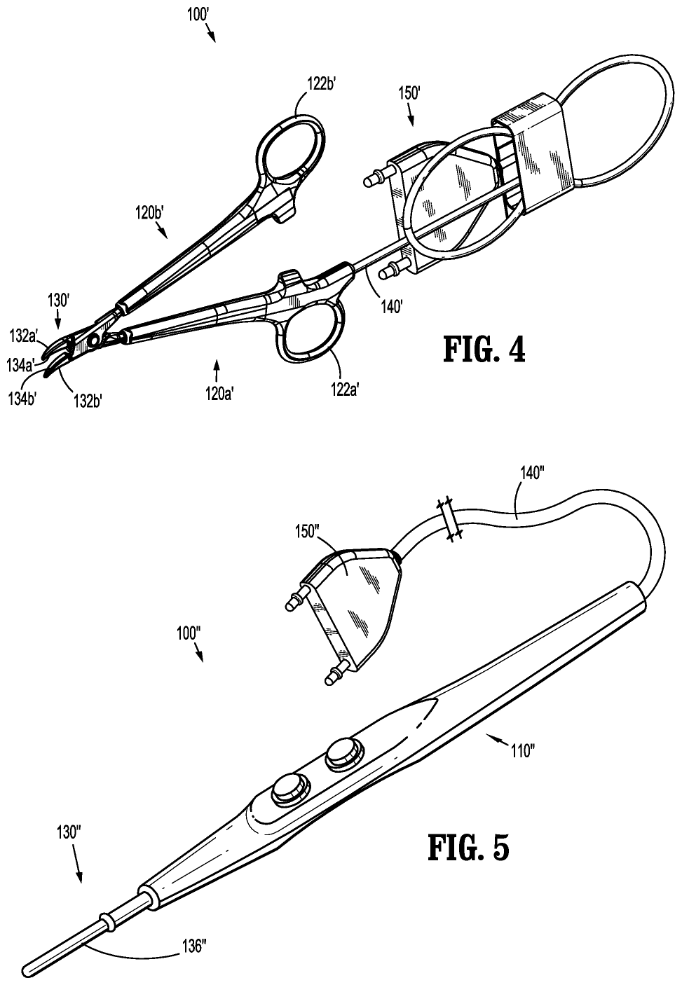

While the electrosurgical instrument 100 is shown as an endoscopic forceps in FIG. 1, the electrosurgical system 10 may additionally or alternatively include other electrosurgical instruments which are electrically connectable to the electrosurgical generator 200, such as those shown in FIGS. 4 and 5, although other instruments including robotic surgical instruments are also contemplated. FIG. 4 illustrates an electrosurgical instrument 100′ in the form of an open forceps which includes first and second elongated shafts 120a′, 120b′ pivotably coupled together. Proximal end portions of the first and second elongated shafts 120a′, 120b′ include first and second handles 122a′, 122b′, and distal end portions of the first and second elongated shafts 120a′, 120b′ cooperate to define an end effector 130′ having opposed first and second jaws 132a′, 132b′ including sealing plates 134a′, 134b′ therein. A cable 140′ extends from the first elongated shaft 120a′ and has a connector plug 150′ at a distal end thereof. FIG. 5 illustrates an electrosurgical device 100″ in the form of an electrosurgical pencil that includes an elongate housing 110″ supporting an end effector 130′ having an electrocautery blade 136″. A cable 140″ extends from the elongate housing 110″ and has a connector plug 150″ at a distal end thereof

The connector plugs 150′, 150″ are configured for insertion into and removal from the plug receptacle 210 (FIG. 1) of the electrosurgical generator 200 in the same or similar manner as the connector plug 150 of the electrosurgical instrument 100 of FIG. 1. Accordingly, it should be understood that the electrosurgical instrument may be endoscopic or open, and the end effector of the electrosurgical instrument may have any suitable type of electrosurgical electrode or electrode assembly for delivering any suitable type of electrosurgical energy, and the plug receptacle of the electrosurgical generator may be configured to accommodate connector plugs of these electrosurgical instruments.

While aspects of the disclosure are discussed in terms of connector systems for interconnecting an electrosurgical instrument to an electrosurgical generator, it is envisioned that the principles of this disclosure are equally applicable to a range of connector systems for electrically interconnecting various instruments and equipment, as well as components thereof, such as removable or replaceable components of a surgical instrument (e.g., an end effector releasably attached an elongated shaft of a surgical instrument).

While aspects of the disclosure have been shown in the drawings, it is not intended that the disclosure be limited thereto, as it is intended that the disclosure be as broad in scope as the art will allow and that the specification be read likewise. It is to be understood, therefore, that the disclosure is not limited to the precise aspects described, and that various other changes and modifications may be effected by one skilled in the art without departing from the scope or spirit of the disclosure. Additionally, the elements and features shown and described in connection with certain aspects of the disclosure may be combined with the elements and features of certain other aspects without departing from the scope of the disclosure, and that such modifications and variation are also included within the scope of the disclosure. Therefore, the above description should not be construed as limiting, but merely as exemplifications of aspects of the disclosure. Thus, the scope of the disclosure should be determined by the appended claims and their legal equivalents, rather than by the examples given.

What is claimed is:

1. A connector system for connecting an electrosurgical instrument to an electrosurgical generator, the connector system comprising:
a connector plug coupled to the electrosurgical instrument, the connector plug including a connector housing and a connector pin extending from the connector housing, the connector pin including an elongated body and a protrusion extending laterally outwardly from the elongated body; and
a plug receptacle defined in the electrosurgical generator, the plug receptacle including an electrical connector disposed therein, the electrical connector including a substrate having a notch defined therein and an electrical contact region disposed thereon, the connector plug removably insertable into the plug receptacle such that the protrusion of the connector pin is positioned within the notch of the electrical connector and the elongated body of the connector pin contacts the electrical contact region of the electrical connector.

2. The connector system according to claim 1, wherein the protrusion of the connector pin is positioned between proximal and distal segments of the elongated body.

3. The connector system according to claim 2, wherein the notch defined in the electrical connector is proximal to the electrical contact region, and the distal segment of the connector pin contacts the electrical contact region when the connector plug is inserted in the plug receptacle.

4. The connector system according to claim 1, wherein the notch and the electrical contact region of the electrical connector are defined on a first surface of the substrate.

5. The connector system according to claim 4, wherein the notch and the electrical contact region are disposed on a planar segment of the first surface, and the first surface includes a tapered segment disposed proximal to the planar segment.

6. The connector system according to claim 1, wherein the protrusion of the connector pin and the notch of the electrical connector have complementary geometries.

7. The connector system according to claim 1, wherein the elongated body of the connector pin is formed from a conductive material and the protrusion is formed from a non-conductive material.

8. The connector system according to claim 1, wherein the electrical connector is a printed circuit board.

9. The connector system according to claim 1, wherein during insertion of the connector plug into the plug receptacle, the protrusion of the connector pin is configured to slide along the substrate of the electrical connector such that the elongated body of the connector pin does not contact the electrical connector until the protrusion aligns with the notch of the electrical connector.

10. An electrosurgical system comprising:
an electrosurgical instrument including a connector plug, the connector plug including a connector housing and a connector pin extending from the connector housing, the connector pin including an elongated body and a protrusion extending laterally outwardly from the elongated body; and
an electrosurgical generator including a plug receptacle, the plug receptacle including an electrical connector disposed therein, the electrical connector including a substrate having a notch defined therein and an electrical contact region disposed thereon, the connector plug removably insertable into the plug receptacle such that the protrusion of the connector pin is positioned within the notch of the electrical connector and the elongated body of the connector pin contacts the electrical contact region of the electrical connector.

11. The electrosurgical system according to claim 10, wherein the protrusion of the connector pin is positioned between proximal and distal segments of the elongated body.

12. The electrosurgical system according to claim 11, wherein the notch defined in the electrical connector is proximal to the electrical contact region, and the distal segment of the connector pin contacts the electrical contact region when the connector plug is inserted in the plug receptacle.

13. The electrosurgical system according to claim 10, wherein the notch and the electrical contact region of the electrical connector are defined on a first surface of the substrate.

14. The electrosurgical system according to claim 13, wherein the notch and the electrical contact region are disposed on a planar segment of the first surface, and the first surface includes a tapered segment disposed proximal to the planar segment.

15. The electrosurgical system according to claim 10, wherein the protrusion of the connector pin and the notch of the electrical connector have complementary geometries.

16. The electrosurgical system according to claim 10, wherein the elongated body of the connector pin is formed from a conductive material and the protrusion is formed from a non-conductive material.

17. The electrosurgical system according to claim 10, wherein the electrical connector is a printed circuit board.

18. The electrosurgical system according to claim 10, wherein during insertion of the connector plug into the plug receptacle, the protrusion of the connector pin is configured to slide along the substrate of the electrical connector such that the elongated body of the connector pin does not contact the electrical connector until the protrusion aligns with the notch of the electrical connector.

19. A method of using an electrosurgical system, the method comprising:
connecting an electrosurgical instrument to an electrosurgical generator by inserting a connector plug of the electrosurgical instrument into a plug receptacle of the electrosurgical generator such that a protrusion of a connector pin of the connector plug is positioned within a notch defined in an electrical connector disposed within the plug receptacle, and an elongated body of the connector pin contacts an electrical contact region of the electrical connector.

20. The method according to claim 19, further comprising:
inserting an end effector of the electrosurgical instrument into tissue; and
actuating an energy activation actuator to control a supply of energy from the electrosurgical generator to the end effector of the electrosurgical instrument.
